(19) United States
(12) Patent Application Publication
(10) Pub. No.: US 2006/0178747 A1
(43) Pub. Date: Aug. 10, 2006

(54) Vertebroplasty method

(76) Inventors: Jan Albert de Vries, Zelhem (NL); James P. Seaton, Chatham, NJ (US); Linda Trebing, Madison, NJ (US)

Correspondence Address: MELVIN I. STOLTZ, ESQ., 51 CHERRY STREET, MILFORD, CT, 06460, US

(21) Appl. No.: 11/393,618

(22) Filed: Mar. 30, 2006

Related U.S. Application Data
(63) Continuation of application No. 11/078,146, filed on Mar. 11, 2005.
(60) Provisional application No. 60/553,100, filed on Mar. 15, 2004.

(51) Int. Cl. A61F 2/44

(52) U.S. Cl. 623/17.16

(57) ABSTRACT

By providing an elastic form stable material which is capable of being delivered directly to a specific desired location within a living creature and provides increased strength and rigidity to the injected location, disorders of the vertebrae of a living creature, for example disorders derived from osteoporosis, are able to be effectively treated. In the preferred method, the elastic form stable material is injected directly into the vertebral body, thereby achieving the desired result.

### BACKGROUND ART

Percutaneous vertebroplasty comprises the injection of bone filler into a vertebral body to be treated via a percutaneous route, usually under X-Ray guidance, such as lateral projection fluoroscopy. The bone filler is injected as a paste or semi-liquid from a suitable gun or injection system via a needle, that has been passed into the vertebral body. Vertebroplasty is usually applied to patients that suffer from osteoporotic fractures, malignant metastatic disease and benign tumors of the bone. The bone filler, once injected, should provide a reinforcement to and improved compressive strength of the vertebral body. In addition to these reinforcing and strengthening properties, it is desirable that the starting bone filler composition is of a viscosity that allows it to flow into the fracture planes.

At present almost all bone fillers are based on polymethyl methacrylate. This type of bone filler is prepared from a mixture of methyl methacrylate powder and a suitable radiopaque agent (opacifier) to which a liquid monomer is added. Then the resulting filler should be injected within a limited period of time, e.g. 5 to 10 minutes before the filler starts to thicken and becomes unworkable. While the filler is being injected, continuous X-Ray imaging is performed in order to watch carefully whether the filler is deposited in the appropriate position and proportion, typically some cubic centimeters, within the vertebral body. The filler thus injected is then cured within the vertebral body to a hard material.

However, this type of bone filler possesses several disadvantages. Principally, due to its hardness and strength, the cured material can easily distort the brittle bone material of the vertebral body into which it has been injected. In addition, this hard and stiff material can act as an abrasive within the pores of the bone, as well as migrate through the softer vertebral bone. Furthermore, there is a risk of leakage from the vertebral body, thereby cutting through or exerting pressure on tissue and nerves. In this regard, the prior art material can be carried into the venous system as a free monomer, leading to the potential occurrence of fatal hypertension episodes. Finally, this material causes a substantial exothermic reaction to occur during the curing process leading to the potential cause of bone necrosis.

Therefore, it is a principal object of the present invention to provide filler material and a method for using the filler material in a vertebral body, or other similar application, which is easily prepared and delivered to the vertebral body while also providing the desired reinforcing and strengthening properties.

Another object of the present invention is to provide filler material and a method for using the filler material in a vertebral body, or similar application having the characteristic features described above which is inherently flexible and viscous to provide flowability throughout the vertebral body both during its application and after curing, thereby achieving self-regulating control.

Another object of the present invention is to provide filler material and a method for using the filler material in a vertebral body, or similar application having the characteristic features described above which is capable of being prepared to exhibit varying levels of hardness or stiffness after curing, thereby allowing the selection and formulation of the filler material with appropriate mechanical properties specifically suited for each application.

Other and more specific objects will in part be obvious and will in part appear hereinafter.

### SUMMARY OF THE INVENTION

By employing the present invention, all of the difficulties and drawbacks of the prior art are eliminated, and a vertebroplasty method of treating disordered vertebral bodies in a living creature, in particular a human being, as well as treating other similar areas of an individual, is attained, along with a composition for formulating an improved filler material. In the present invention, the method comprises the step of injecting a curable bone filler composition in the vertebral body, wherein said curable bone filler comprises an elastic form stable material.

According to the present invention, a creature such as a human being in need of a vertebroplasty treatment, e.g. derived from osteoporosis, is treated by reinforcing and strengthening the particular vertebral body or bodies. To this purpose a bone filler is injected into the selected vertebral body, wherein the bone filler is an elastic form stable material. Due to its properties with respect to elasticity and dimensional stability, the forces exerted on and within the brittle vertebral body upon and after injecting are smaller, thereby reducing the risk of breaking or disrupting the body thus treated. After curing, this non-resorbable material is a solid but still flexible mass that is not likely to migrate through the body, but holds its initial position where it is applied.

The method and filler material of the present invention, as detailed herein, has wide applicability both to vertebral bodies and other areas or sites in an individual wherein support and/or reinforcement of bones or joints are required due to diseases, accidents, and/or injuries. Typical of such difficulties are various compressive fractures, as well as degenerative diseases of the hip.

Furthermore, other treatment methods, wherein reinforcing elements are inserted in a vertebral body or means for treating or removing bone fragments, can benefit from the present invention. In this regard, using the method and composition of the present invention, void or open areas resulting from the use of these reinforcing systems, can be filled, thereby eliminating areas of weakness.

Consequently, although the following detailed discussion focuses upon the use of the present invention in connection with vertebral bodies, it is to be understood that this invention is equally applicable to all other areas of the body wherein the benefits provided by the present invention are equally applicable.

Typically the vertebroplasty method according to the invention comprises the following steps. The patient is examined usually by CT and/or MR imaging in order to provide the data upon which a decision about the vertebral body to be treated is made. The patient to be treated is positioned in a prone position, and the skin covering the vertebral body is prepared. The patient is anesthetized, usually by locally injecting a suitable anesthetic into the skin underlying fat and into the periosteum of the pedicle to be entered.

Next, a skin incision is made using a scalpel or other surgical instrument. A needle having a suitable gauge is selected and passed down the pedicle into the respective vertebral body. Alternatively, the needle or catheter may be introduced through the posterior-lateral aspect of the vertebral body. If desired, any unwanted leakage can be detected by injecting a suitable X-Ray contrast liquid into the vertebral body and performing a vertebrogram.

Meanwhile, the filler is prepared and is then injected, while monitoring the position of the needle, and the amount of injected filler material, e.g. by lateral X-Ray projection fluoroscopy imaging. For that purpose a suitable opacifier is added to the bone filler. The injection is stopped when the filling of the vertebral body has been completed to the desired extent. If the filler starts to flow into unwanted locations, injection will also be interrupted.

In the method according to the invention, the elastic form stable material preferably comprises an elastomer-precursor composition which is cured in situ. That is to say, the elastomer precursor is prepared in advance and then applied to the appropriate vertebral body by suitable equipment.

Furthermore, the filler material of the present invention is substantially less toxic than the prior art filler material. Consequently, the filler material of this invention is tolerated more easily by patients and, as a result, may be used prophylactically to prevent or reduce problem areas which have not reached a severe or critical level.

The invention also relates to a method of preparing a composition for treating a disordered vertebral body in a living creature, in particular a human being, comprising an elastic form stable material. Usually this method comprises the steps of providing the starting materials of the elastic form stable materials in the appropriate ratio into separate containers (or separated chambers that may be present in one mixing/dispensing device), and packaging thereof.

The invention accordingly comprises the several steps and the relation of one or more of such steps with respect to each of the others, and the composition possessing the features, properties, and the relation of ingredients, which will be exemplified in the composition hereinafter described, and the scope of the invention will be indicated in the claims.

### THE DRAWINGS

For a fuller understanding of the nature and objects of the present invention, reference should be held to the following detailed description taken in connection with the accompanying drawings, and which:

FIG. 1 is a cross-sectional side elevation view depicting three adjacent vertebral bodies, with the composition of the present invention in the process of being used in treating one of the vertebral bodies.

### DESCRIPTION OF THE PREFERRED EMBODIMENTS

According to the preferred embodiment of the present invention, the vertebroplasty composition comprises a curable elastomer-precursor composition. Commercially available medical grade silicone elastomers are preferred materials for use as polymer precursor in this composition. A more preferred material is poly (dimethyl siloxane) such as hydroxyl-end-blocked poly (dimethyl siloxane). Such silicone elastomers of medical grade as pourable, multi-component silicones are commercially available.

For these types of elastomers, propyl orthosilicate is a useful cross-linking agent. Fillers and diluents (medicinal fluids such as known under the trade name Dimeticonum) in order to reduce viscosity may be added as needed. It is preferred that a well flowable composition is used, so that even small cracks are filled with the composition.

In the preferred formulation, an initiator like tin (II) octoate initiates the polymerization reaction with splitting of propanol. The reaction proceeds without the generation of sensible heat, which is also beneficial to the patient treated. Silphenylene polymer can be used in a similar way.

In order to be able to trace the position of the cured applied composition by X-Ray monitoring, a preferred embodiment of the composition of the present invention, comprises a radiopaque material, such as silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents. In this regard, silver powder is the preferred radiopaque material, since smaller proportions are needed in the composition and the material also provides a material anti-bacterial and anti-microbial activity.

A composition as explained above is known, per se, as a material suitable for the non-surgical, reversible sterilization of females. In this known sterilization method, the composition is injected in the oviduct portion adjacent the uterus, where it forms in situ a block or plug in the oviduct, thereby preventing the passage of ovum from the ovaries to the uterus and sperm from entering the oviduct and thus conception. See U.S. Pat. No. 4,245,623. However, the present is patentably distinguished from this prior art.

A preferred composition for use in the method according to the invention comprises about 60-85% by weight poly (dimethyl siloxane), about 2-5% cross-linking agent, a diluent in the range of 10-20%, and about 10-20% radiopaque powder.

In the method according to the invention the composition is advantageously prepared in advance in a mixing-dispensing device. Such a device, wherein the function of mixing the components is combined with the function of dispensing the thus prepared mixture, is known per se, e.g. from the above U.S. Patent, the content of which is incorporated in its entirety by reference. In addition, other mixing and dispensing systems known in the art.

One of these alternate, two-component mixing and dispensing devices is fully disclosed in pending U.S. patent application Ser. No. 11/000,578, filed Nov. 30, 2004, entitled Two Component Mixing and Dispensing Device. If desired, the composition and method of the present invention may be employed using this device and the pertinent disclosure of this pending patent application is incorporated herein by reference.

As already indicated above, the invention also relates to a method of preparing an injection composition for injection into a disordered vertebral body of a living creature, in particular a human being, said composition comprising an elastic form stable material. The above-mentioned preferred features of the treatment method according to the invention are similarly applicable to the preparation method according to the invention.

Advantageously the injection composition is packaged as a kit of parts, comprising at least a first container filled with an elastomer precursor and optionally a diluent, and a second container filled with a cross-linking agent for this elastomer precursor. More preferably, the composition is packaged in a mixing-dispensing device, comprising such containers with a temporary seal between the containers. In addition, one of these containers is provided with a stirrer which can be operated manually or powered by an external source.

An example of such a device is also known from the above-mentioned U.S. Patent. Devices of this type can be used for injecting the thus prepared precursor composition by connecting a suitable flexible tube to the container acting as a mixing chamber and providing an appropriate needle at the other end of the tube.

In the invention a flowable composition is prepared from the various components, preferably in a combined mixing-dispensing device as explained above, and then immediately used. In order to assist in accurately positioning the deposit of the composition, the needle employed typically has an open tip, and may have one or more exits on the side face directly adjacent to the tip. Once the vertebral body is filled, the needle is retracted and the composition is allowed to cure in situ. If necessary, the above actions can be repeated as needed.

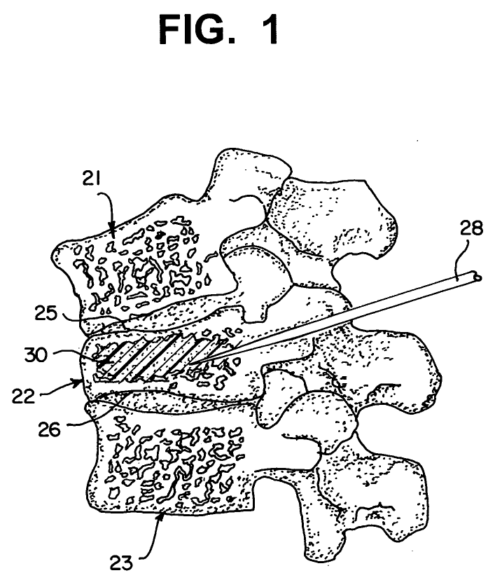

In order to best understand the method of this invention, reference should be made to FIG. 1, along with the following disclosure. In FIG. 1, three adjacent vertebral bodies 21, 22, and 23 are depicted with varying stages of osteoporosis. As shown, vertebral body 22 has the most serious problem, with end plates 25 and 26 thereof angularly pitched towards each other. In order to prevent any further deteriorations of vertebral body 22, the composition and method of the present invention is employed.

In using the present invention, the elastic form stable, curable bone filler material detailed above is prepared and is readied for delivery to vertebral body 22. Needle 28 is inserted into the individual, with the tip thereof inserted into vertebral body 22. Then, filler material 30 is forced through needle 28, directly into the cavity of vertebral body 22.

Due to the controlled viscosity of filler material 30, filler material 30 is able to freely flow through the bony structure of vertebral body 22, completely filling the cavity thereof. Once the desired area has been completely filled, the flow of filler material 30 is stopped and needle 28 is removed.

Once the process is complete, filler material 30 is allowed to cure in situ, thereby providing vertebral body 22 with a solid, flexible mass which is able to strengthen and reinforce the vertebral body and prevent further degradation thereof. Furthermore, due to inherent viscosity of filler material 30 after curing, filler material 30 is also able to provide a self-adjusting movement or shifting within the vertebral body 22, providing strength and reinforcement to any area or zone where such reinforcement or strength is needed.

In addition, the durometer of filler material 30, when cured and retained in the enclosed area defined by vertebral body 22, enables filler material 22 to provide inherent strength and rigidity wherever required, while being compressible and eliminating distortion and/or breakage of brittle bone material in the vertebral body.

Furthermore, filler material 30 may be formulated to cure into a particularly desired durometer which may vary over a wide range. In general, the cured durometer of filler material 30 can be formulated to range between about 10 A and 70 A.

By controlling the durometer of cured filler material 30, a customized result can be achieved wherein filler material 30 is selected and formulated for treating a particular pathology in the optimum manner. Also, filler material 30 is relatively soft and compressible, thereby allowing the vertebral body to interact with the adjacent vertebral bodies and discs by absorbing shocks or impacts, reducing possible damage to the surrounding elements.

Although FIG. 1 depicts the addition of filler material 30 to only vertebral body 22, the same method can be employed to deliver filler material 30 to any other vertebral body. Consequently, if vertebral body 21 and/or 23 were damaged and in need of repair, the same process could be employed to deliver filler material 30 to these vertebral bodies, or any other area in need of repair.

It will thus be seen that the object set forth above, among those made apparent from the preceding description, are efficiently attained and, since certain changes may be made in carrying out the above method and in the composition set forth without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Particularly, it is to be understood that in said claims, ingredients or compounds recited in the singular are intended to include compatible mixtures of such ingredients wherever the sense permits.

Having described our invention, what we claim as new and desire to secure by Letters Patent is:

1. A vertebroplasty method for treating a disordered vertebral body in a living creature, in particular a human being, comprising the step of injecting a curable bone filler composition in said vertebral body, wherein the curable bone filler material comprises an elastic form stable material.

2. The method defined in claim 1, wherein said composition comprises a curable elastomer-precursor composition.

3. The method defined in claim 2, wherein said composition comprises a silicone elastomer.

4. The method defined in claim 3, wherein said silicone elastomer comprises poly (dimethyl siloxane).

5. The method defined in claim 4, wherein said composition additionally comprises a cross-linking agent and a diluent.

6. The method defined in claim 5, wherein said composition comprises a radiopaque material.

7. The method defined in claim 6, wherein said radiopaque material comprises one selected from the group consisting of silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents.

8. The method defined in claim 7, wherein curable bone filler material is further defined as comprising:
A. between about 60% and 85% by weight based upon the weight of the entire composition of poly (dimethyl siloxane);
B. between about 2% and 5% by weight based upon the weight of the entire composition of the cross-linking agent;
C. between about 10% and 20% by weight based upon the weight of the entire composition of the diluent; and
D. between about 10% and 20% by weight based upon the weight of the entire composition of the radiopaque material.

9. The method defined in claim 8, wherein said composition is prepared in advance in a mixing-dispensing device.

10. The method defined in claim 8, wherein said composition is delivered to the vertebral body by inserting a needle into the internal cavity of the vertebral body and causing the filler material to flow through the needle into the vertebral body.

11. The method defined in claim 10, comprising the additional steps of stopping the flow of the filler material when the vertebral body has been filled with the filler material, and thereafter withdrawing the needle from the vertebral body.

12. The method defined in claim 8, wherein the curable bone filler is formulated to possess a durometer in the cured state which ranges between about 10 A and 70 A.

13. The method defined in claim 18, wherein said filler material is further defined as being flexible, when cured, to move, shift, compress, and or elongate within the porous structure of the vertebral body, thereby providing varying actions or reactions.

14. A method of preparing a composition for injection into a disordered area of the body of a living creature, in particular a human being, said composition comprising an elastic form stable material consisting of a curable elastomer-precursor composition and additives intermixed therewith and possesses substantially reduced toxicity, thereby enabling its use prophylactically.

15. The method defined in claim 14, wherein the curable elastomer-precursor composition comprises a silicone elastomer.

16. The method defined in claim 15, wherein said silicone elastomer is poly (dimethoxy siloxane).

17. The method defined in claim 16, wherein the additives of said composition comprises a cross-linking agent, a diluent, and a radiopaque material.

18. The method defined in claim 17, wherein said composition is packaged as a kit of parts, comprising filling a first container with said silicone elastomer, and filling a second container with said cross-linking agent.

19. The method defined in claim 18, wherein said kit of parts comprises a mixing-dispensing device, comprising said container and a temporary seal between the containers, wherein one container is provided with a movable stirrer.

20. The method defined in claim 18, wherein said kit of parts comprises a mixing-dispensing device, comprising said containers and a mixing channel at the exits of the two-containers, wherein said channel is provided with a static mixing element.

21. The method defined in claim 17, comprising the steps of thoroughly intermixing:
A. between about 60% and 85% by weight based upon the weight of the entire composition of poly (dimethyl siloxane);
B. between about 2% and 5% by weight based upon the weight of the entire composition of the cross-linking agent;
C. between about 10% and 20% by weight based upon the weight of the entire composition of the diluent; and
D. between about 10% and 20% by weight based upon the weight of the entire composition of the radiopaque material,
thereby forming the desired inject composition.
